(19) United States
(12) Patent Application Publication
(10) Pub. No.: US 2012/0071884 A1
(43) Pub. Date: Mar. 22, 2012

(54) MIXING AND DISPENSING APPARATUS FOR BONE VOID FILLER

(75) Inventors: John Joseph COOPER, Crewe (GB); Michael James BRAYFORD, Newcastle-under-Lyme (GB); David Russell WATERS, Audlem (GB)

(73) Assignee: BIOCOMPOSITES LIMITED, Keele (GB)

(21) Appl. No.: 13/220,627

(22) Filed: Aug. 29, 2011

(30) Foreign Application Priority Data
Sep. 1, 2010 (GB) 1014514.2

(51) Int. Cl. A61B 17/58; B65D 83/00; B28C 7/14; C04B 28/14; C04B 12/02

(52) U.S. Cl. 606/93; 106/772; 106/690; 366/6; 206/216

(57) ABSTRACT

A method for preparing a de-aired hydraulic setting hardenable bone cement for filling a boney void or cavity by combining a powder bone substitute material and a aqueous liquid component, the method comprising the steps of: (a) supplying said powder bone substitute material in a first evacuable container; (b) withdrawing air from the first container to form a de-aired powder bone substitute material; and, then (c) mixing the de-aired powder bone substitute material and the aqueous liquid component together.

### BACKGROUND OF THE INVENTION

The present invention provides an apparatus and method for producing a de-aired hydraulic setting hardenable bone cement (i.e. a de-aired mixture comprising a bone substitute material and an aqueous liquid component which hardens/fully sets to form a bone cement due to the chemical reaction of the bone substitute material with the aqueous liquid component) suitable for the treatment of bone disorders and filling of boney voids or defects of the skeletal system of the human or animal body. In particular, although not exclusively, the present invention provides a simple means to produce a de-aired hydraulic setting hardenable bone cement in a sterile manner which may be dispensed directly to the surgical site in a convenient and minimally invasive manner, whereby it hardens in-situ to form a de-aired bone cement having desirable mechanical strength and fatigue resistance.

Bone cements in the form of grafts and bone void fillers are used in orthopaedic and dental surgery for filling voids or defects of the skeletal system. These defects may be surgically created osseous defects or osseous defects resulting from disease or traumatic injury to the bone. The bone cements may have a wide range of differing chemical formulations and may be prepared from mixtures having different physical and rheological properties e.g. the mixture may be in the form of a liquid or a paste.

Poly-methyl methacrylate (PMMA) bone cement has been used in cemented hip joint replacement procedures for many decades. PMMA bone cements are non-hydraulic setting bone cements (i.e. a cement which is not formed by the chemical reaction of one component with an aqueous component). PMMA cements are typically prepared from essentially liquid formulations (i.e. a formulation which does not support a shear force) comprising a mixture of liquid components, and optionally a small amount of a particulate filler component, such as barium sulphate for enhanced X-ray opacity. The liquid formulation comprising the mixture of liquid components may be mixed in vacuo to remove trapped air bubbles from the mixture prior to use/setting as a bone cement. Such a procedure typically involves the use of a closed container in which the components are mixed together to form a mixture which essentially behaves as a liquid. A vacuum is then applied to the mixture which causes entrapped air bubbles to expand and rise to the surface where they are expelled along with the toxic vapors from the cementitious PMMA material. The use of such vacuum mixing systems to remove entrapped air when preparing PMMA bone cement may reduce its porosity and thereby improve the consistency, homogeneity, fatigue properties and strength of the resulting cement.

Alternative hydraulic setting bone cements are also known and typically comprise a calcium salt based bone substitute material. These materials when implanted into the body are intended to resorb and be replaced by new bone as they do so. They may consist of a range of different calcium based salts incorporating sulphate and/or phosphate based anions. The calcium based bone substitute material is typically supplied in the form of a powder which when mixed with an aqueous liquid component (e.g. an aqueous solution or water) initiates a chemical reaction and forms a hardenable cohesive mass (i.e. a paste); the hardenable cohesive mass cures and finally sets to form a calcium based bone cement. When used as a bone graft material they are typically employed as an aqueous paste comprising a highly filled suspension of particulate components (i.e. the calcium based bone substitute material) in an aqueous medium which is applied either digitally packed or injected to the surgical site where it then sets in-situ.

Calcium sulphate hemi-hydrate is one such calcium based bone substitute material which may be employed to form a hydraulic setting bone cement. When the calcium sulphate hemi-hydrate powder is blended with an appropriate quantity of water or aqueous solution (e.g. salt solution), the mixture hydrates to form a cohesive mass and sets with a mildly exothermic reaction to give calcium sulphate di-hydrate (i.e. the bone cement) according to the following reaction:

CaSO₄½H₂O (Plaster of Paris)+1½H₂O=CaSa₄2H₂O (gypsum)

The calcium sulphate hemi-hydrate may be used alone or it may be used in combination with a calcium phosphate based bone substitute material.

Calcium phosphate cement (CPC) is another type of hydraulic setting bone cement which may be used as a synthetic bone graft material. The original cement, devised by L. C. Chow and W. E. Brown in 1986, is formed from a calcium phosphate based bone substitute material comprising a white powder consisting of equimolar amounts of ground Ca₄(PO₄)₂O (tetracalcium phosphate, TTCP) and CaHPO₄ (dicalcium phosphate anhydrous, DCPA). The powder when mixed with water forms a workable paste which can be shaped during surgery to fit the contours of a wound. The paste hardens (i.e. fully sets) in-situ within 20 min to form the calcium phosphate cement, thereby allowing rapid closure of the wound. The hardening reaction, due to reaction of TTCP and DCPA with the water, forms nanocrystalline hydroxyapatite (HA) as the main product; it is isothermic and occurs at physiologic pH so tissue damage does not occur during the setting reaction.

There are now available a number of other different formulations of calcium phosphate hydraulic setting bone cements (CPCs). These may contain a range of calcium based salts including monocalcium phosphate, dicalcium phosphate, tetracalcium phosphate, octacalcium phosphate and calcium carbonate and combinations thereof. The calcium based salts are mixed together with an aqueous liquid, which may also contain soluble phosphates such as sodium phosphate or phosphoric acid, to form an aqueous paste which after application to the surgical site sets in-situ to form a bioabsorbable hydroxyapatite or carbonated apatite having a chemical and crystallographic similarity to the apatite in human bones and as such represent highly promising materials for clinical applications.

As with PMMA cements, it would be advantageous to provide a hydraulic setting bone cement in a de-aired condition for dispensation to the surgical site in the hope that such a cement may exhibit improved mechanical strength and fatigue resistance. Additionally, for certain clinical applications where the material is injected into a boney void under pressure, such as vertebroplasty or kyphoplasty procedures, it is desirable to remove air from the paste prior to injection to reduce the risk of possible embolisms.

It has been found that known evacuation systems for de-airing cementitious PMMA materials are typically unsuitable for applications involving hydraulic setting bone cements as the cements are formed from an aqueous cohesive paste comprising a highly filled suspension of particulate components in an aqueous medium. The cohesive paste has a relatively high viscosity and exhibits properties of a gel or thioxotropic solid, rather than of a liquid as in PMMA based materials. Consequently, it has been found that the expanded air bubbles formed in the cohesive paste by application of a vacuum may remain trapped within the paste rather than rise freely to the surface. Although it may be possible to dislodge some of the larger air bubbles by shaking or mixing the mixture; removal of the smaller air bubbles is more problematic. Moreover, mixing the mixture may also cause the entrapped air bubbles to be broken-up into smaller bubbles. Furthermore, as the cohesive paste progressively sets during the procedure, the viscosity of the paste continuously increases thereby making removal of the entrapped air bubbles even more problematic with the passage of time.

The present invention therefore seeks to provide a method and apparatus for producing a de-aired hydraulic setting hardenable bone cement material.

The present invention also seeks to provide a method and apparatus for producing a de-aired hydraulic setting hardenable bone cement material in a sterile manner which can be dispensed directly to the surgical site in a convenient and minimally invasive manner, whereby it cures/sets in-situ to form a de-aired bone cement.

### SUMMARY OF THE INVENTION

Thus in accordance with a first aspect, the present invention provides a method for preparing a de-aired hydraulic setting hardenable bone cement for filling a boney void or cavity by combining a powder bone substitute material and an aqueous liquid component, the method comprising the steps of:
(a) supplying said powder bone substitute material in a first evacuable container;
(b) withdrawing air from the first container to form a de-aired powder bone substitute material; and, then
(c) mixing the de-aired powder bone substitute material and the aqueous liquid component together.

Advantageously, it has been found by preparing a hydraulic setting bone cement where the aqueous liquid component is added to a previously de-aired powder component typically provides a bone cement having improved mechanical strength, increased density and fatigue resistance. Conveniently, such cements may exhibit improved X-ray visibility and be resorbed more slowly in vivo. Additionally, such a de-aired hydraulic setting hardenable cement being devoid of air is more suitable for injection into a boney void under pressure (e.g. vertebroplasty or kyphoplasty procedures), as it is less likely to induce an embolism.

Suitably, the first evacuable container retains its structural integrity when air is withdrawn from the container during step (b) of the method of the first aspect of the invention. Advantageously, this prevents the powder bone substitute material (i.e. the free flowing powder bone substitute material) from forming a densified powder compact which is more difficult to hydrate with the aqueous liquid component in step (c). Preferably, the first evacuable container comprises a syringe having a lockable piston and barrel construction.

Preferably, the aqueous liquid component is provided in a second separate container. More preferably, the second container is capable of retaining its structural integrity when air is withdrawn therefrom. Even more preferably, the second container comprises a syringe having a piston and barrel construction, wherein the piston of the syringe is free to move within its associated barrel.

Preferably, the mixing step (c) is performed by connecting the first and second containers together in fluid communication. Suitably, when the first container comprises a first lockable syringe, the piston of the lockable syringe is kept in a locked position during the de-airing step (b) and when the second container including the aqueous liquid component is initially connected in fluid communication with the lockable syringe. Consequently, when the second container comprises a second syringe, the barrel of the second syringe advances as the aqueous liquid exits therefrom into the first locked syringe by virtue of the lower pressure state in the first syringe, thereby promoting homogeneous mixing of the aqueous liquid component with the de-aired powder bone substitute material. Following ingress of the aqueous liquid from the second syringe into the first syringe, the piston of the first syringe may be unlocked so that it is free to reciprocate within its associated barrel. Homogeneous mixing of the de-aired hydraulic setting hardenable bone cement may be effected by alternately depressing the plungers of the first and second syringes to cause the hardenable cement to pass between both syringes and progressively homogenize the mixture into a paste. Conveniently, when the de-aired hydraulic setting hardenable bone cement is judged to be sufficiently mixed and of a suitable paste like consistency, the hardenable bone cement may be passed into the first or second syringe and it may be dispensed directly to a surgical site where it sets in-situ to form a de-aired bone cement.

The mixing of the de-aired bone substitute material and the aqueous liquid component may be performed at least partially within the first container under “no touch” conditions, whereby the components are at all times kept isolated from external contamination during steps (b) and (c). Advantageously, such an arrangement permits the formation of a de-aired hydraulic setting hardenable bone cement under sterile conditions.

Preferably, in step (b) of the method of the first aspect of the invention, air is withdrawn from the first evacuable container via a multi-port connector having a first port which is connected to an outlet of the first container and a second port which is connected to a vacuum source, preferably via an air bleed line. More preferably, the aqueous liquid component is supplied in a second container and the multi-port connector has a third port which is connected to receive selectively the aqueous liquid component from an outlet of the second container. Suitably, during step (b) the outlet of the second container is blocked, whilst the outlet of the first container is connected to the vacuum source via the multi-port connector. After the first container has been evacuated to a desired low pressure state the connection between the vacuum source and the first container is closed via the multi-port connector to maintain the first container in a reduced pressure state. The first container in the reduced pressure state may then be connected to the second container in the relatively higher pressure state via the multi-port connector, thereby enabling the liquid component to be drawn into the first container. Optionally, the multi-port connector may include a further port connectable to atmospheric pressure to facilitate easy removal of the vacuum source.

Suitably, the multi-port connector is configurable for one or more of the following states:
(a) to connect an outlet of the first container to the source of vacuum to permit a reduction of the pressure within the first container to a given level below atmospheric pressure;
(b) to isolate the outlet of the first container from the source of vacuum thereby maintaining the first container in a reduced pressure state prior to connecting the outlet of the second container to the first container;
(c) to connect an outlet of the first container to an outlet of the second container, thereby permitting the aqueous liquid component to flow from the second container into the first container;
(d) to permit mixing of the de-aired powder bone substitute material and the aqueous liquid component within it, particularly by selective manipulation of piston plungers; and,
(e) to permit disconnection of the first container from the second container when the de-aired hydraulic setting hardenable bone cement is judged to be sufficiently mixed and of a suitable paste like consistency in the first container.

Suitably, the powder bone substitute material as defined herein after is in the form of a free flowing powder. Suitably, the free flowing powder has an average maximum particle size as measured by X-ray diffraction of 150 microns or less, preferably between 10 to 150 microns.

Suitably, the aqueous liquid component comprises water or an aqueous solution, for example an aqueous salt solution.

According to a second aspect, the present invention provides a method of filling a boney void or cavity in the skeletal system of a human or animal body, the method comprising forming a de-aired hydraulic setting hardenable bone cement in accordance with the first aspect of the invention, dispensing the de-aired hydraulic setting hardenable bone cement from said first and/or second container into a boney void or cavity of a human or animal body and allowing the de-aired hydraulic setting hardenable bone cement to set fully in-situ to form a bone cement.

According to a third aspect, the present invention provides apparatus for preparing a de-aired hydraulic setting hardenable bone cement according to the method of the first aspect of the present invention. Preferably, the apparatus is as defined in accordance with the method of the first aspect of the invention. More preferably, the apparatus is supplied in the form of a sterile kit of parts to an end user.

According to a fourth aspect, the present invention provides a de-aired hydraulic setting hardenable bone cement obtainable by the method according to the first aspect of the present invention

In this specification, the following words and expressions, if and when used, have the meanings ascribed below:

“hydraulic setting hardenable bone cement” means a composition comprising a mixture of a bone substitute material as defined herein and an aqueous component (e.g. water or an aqueous liquid solution) which sets (i.e. fully hardens) to form a bone cement by virtue of the chemical reaction between the bone substitute material and the aqueous component in the mix. Typically, the hydraulic setting hardenable bone cement is in the form of a workable paste;

“bone cement” means a cement formed from a fully hardened/set hydraulic setting hardenable bone cement (i.e. the ultimate end product). The bone cement is suitable for the treatment of bone disorders and filling of bony voids or defects of the skeletal system;

“bone substitute material” means a bioabsorbable material which may be used for the treatment of bone disorders and filling bony voids or defects of the skeletal system to permit regeneration of natural bone growth in the skeletal system and which is capable of forming a bone cement by the chemical reaction with water or an aqueous solution. Preferably, the bone substitute material comprises a calcium salt based material;

“comprising” or any cognate word specifies the presence of stated features, steps, or integers or components, but does not preclude the presence or addition of one or more other features, steps, integers, components or groups thereof. The expressions “consists of” or “consists essentially of” or cognates may be embraced within “comprises” or cognates, wherein “consists essentially of” permits inclusion of substances not materially affecting the characteristics of the composition to which it applies.

### BRIEF DESCRIPTION OF THE DRAWINGS

The various features of the invention, which are applicable as appropriate to all aspects, will now be described in more detail with reference to the following drawings, where:

FIGS. 1a and 1b shows a kit of parts comprising for preparing hydraulic setting bone void fillers;

FIG. 2 shows the apparatus of FIG. 1 in a state for de-airing of a powder component;

FIG. 3 shows the apparatus in a state in which the powder component has been de-aired and mixing initiated; and

FIG. 4 shows a variant of the apparatus in which a multi-port connector part is supplied to an end user pre-connected to the liquid container.

### DETAILED DESCRIPTION

### Bone Substitute Material

Preferably, the bone substitute material comprises a calcium salt based bone substitute material. The bone substitute material is in the form of a powder, preferably a free flowing powder. The bone substitute material when mixed with an aqueous liquid component, for example an aqueous solution (e.g. aqueous salt solution) or water, forms a workable paste (i.e. a hydraulic setting hardenable bone cement) which on setting/curing forms a hardened solid bone cement. Suitable calcium salt based bone substitute materials include calcium sulphates, calcium phosphates, calcium carbonates and combinations thereof. Preferably, the bone substitute material comprises at least one calcium sulphate, especially calcium sulphate hemihydrate which when mixed with water sets with a mildly exothermic reaction to produce solid calcium sulphate dihydrate.

The at least one calcium based bone substitute material may be used alone or it may be used in combination with one or more calcium phosphate bone substitute materials or calcium carbonates.

Examples of suitable calcium phosphate bone substitute materials include monocalcium phosphate, dicalcium phosphate, tricalcium phosphate, tetracalcium phosphate, octacalcium phosphate or hydroxyapatite.

In accordance with a preferred embodiment of the present invention, the bone substitute material comprises a mixture of a calcium sulphate and calcium phosphate bone substitute materials, particularly a mixture of calcium sulphate hemihydrate and tricalcium phosphate, especially beta-tricalcium phosphate.

The bone substitute material, and hence the resulting bone cement, may include a therapeutically active agent. Suitable therapeutically active agents include: bone inducing growth factors to accelerate bone growth such as bone morphogenetic proteins and parathyroid hormones; bone breakdown inhibitors such as biphosphonates and osteocalcin; compounds to prevent invasion by foreign living material such as antibiotics, antibacterial compounds, antiviral compounds and antifungal compounds; and anti-inflammatory compounds such as non-steroidal anti-inflammatory compounds (NSAIDs). A highly preferred therapeutically active agent comprises an antibiotic.

Alternatively, or additionally, the bone substitute material, and hence resulting bone cement, may include an agent to enhance visualisation of the bone cement in-vivo. Suitable agents include ionic and non-ionic X-ray contrast agents, preferably non-ionic water soluble X-ray contrast agents, such as iodine based media e.g. isohexyl.

The therapeutically active agent and/or visual enhancement agent may be included in powder form together with the bone substitute material prior to mixing with the aqueous liquid component. Alternatively, or additionally, the therapeutically active agent and/or visual enhancement agent may be dissolved or dispersed in the aqueous liquid component for mixing with the bone substitute material.

### Vacuum Mixing and Dispensing System

### Example 1

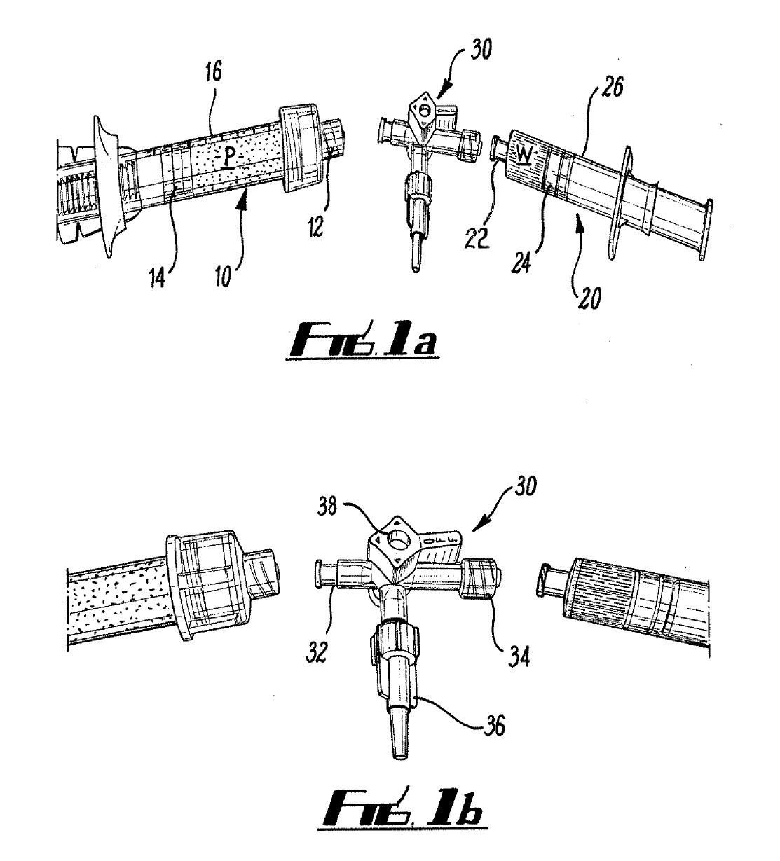

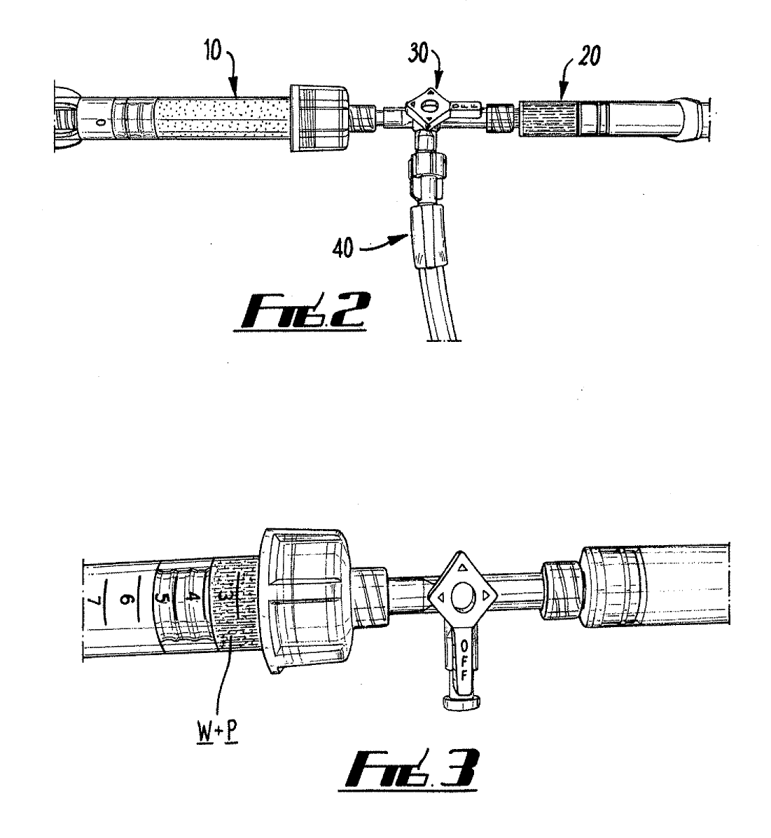

Referring to FIGS. 1a and 1b there is shown apparatus for preparing a de-aired hydraulic setting hardenable bone cement (i.e. bone void filler). The apparatus comprises a first and second container and a multi-port connector.

In this example, five grams of calcium sulphate alpha hemi-hydrate powder, P, having a particle size of substantially less than 150 microns is contained in the first container, which comprises a first piston syringe 10. The first piston syringe 10 has a 10 ml capacity and features a male luer-lock connector 12 and a lockable piston 14 within syringe barrel 16. The first syringe is typically supplied to an end user with an end cap in place over the luer-lock to maintain the contents of the syringe to be free of contamination.

The second container comprises a second piston syringe 20 and this has a female luer-lock connector 22 and a piston 24 moveable within syringe barrel 26. The syringe 20 has a 5 ml capacity and contains 1.7 ml of water, W.

The multi-port connector comprises a “T” piece connector 30 which is a 3-way tap having a female luer-lock connector 32, a male luer-lock connector 34, a nozzle 36 and a turnable tap 38 for selectively connecting any two of the three ports together or for isolating them. The two luer-locks 32, 34 and the corresponding luer-lock connections 12, 22 on the first and second syringes ensure that the first and second syringes may be attached to the 3-way tap in an airtight manner.

Referring now to FIG. 2, bleeding of air from the powder contained in the first syringe 10 and the subsequent mixing of the powder with water from the second syringe 20 will now be described.

Typically all three components (the two syringes and the tap connector) are provided to the user in a sterile package with luer-lock end caps on the syringes to maintain substance integrity right up to the point where the user makes the connections between them.

In FIG. 2, there is shown the first and second syringes 10, 20 connected to the 3-way tap connector 30 by means of the luer-lock connections and there is shown an air bleed line 40 attached to the nozzle 36 and a vacuum source (not shown).

The piston 14 of the first, powder, syringe 10 is provided initially in the locked condition. The position of the piston 14 in the barrel 16 of the syringe 10 is such that the loose-fill powder mass occupies approximately half (or less) of the available volume within the syringe barrel 16. This facilitates the application of a vacuum to the first syringe 10, through the three-way connector 30, without the syringe piston 14 moving within the syringe barrel 16 and also ensures that the powder does not impede air removal by evacuation. The three-way connector 30 in a first condition has a flow-through from the nozzle 36 (i.e. the vacuum arm) of the connector 30 to the powder syringe 10 only.

The air bleed line 40 is attached to the third arm of the three-way connector 30 by means of a simple push-on connector. Vacuum is then applied to the powder with the powder contents of the syringe 10 lying horizontal in the lower half of the syringe barrel 16 to enable unimpeded exhaust of the air from the syringe barrel without egress of the powder from the barrel 16 to the air bleed line 40. The vacuum pressure may be monitored by a suitable vacuum gauge, such as a Bourdon type gauge, and the vacuum is applied for sufficient time to enable removal of air from within the powder mass. A suitable level of vacuum would be a pressure of less than 0.2 bar and preferably less than 0.1 bar and more preferably less than 0.05 bar.

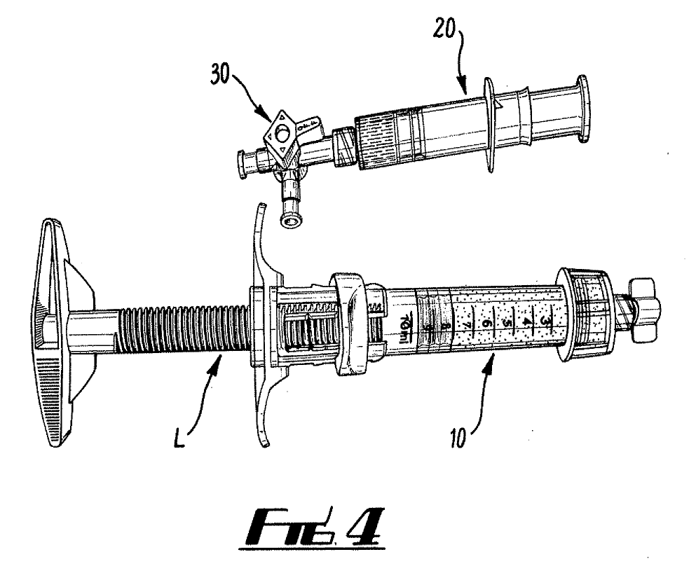

Following the de-airing step the tap 38 of the three-way connector 30 is rotated to close the air bleed line 40 which can now be disconnected. The connector 30 is then configured to open the connection between the first syringe 10, containing the powder, and the second syringe 20 containing the water. Immediately this is done the lower pressure state of the first syringe as compared to the pressure within the second syringe and the connecting pathway through the connector 30 causes the water in the second syringe 20 to be drawn into the powder as shown in FIG. 3. The piston 24 and the associated plunger in the water containing second syringe 20 advances as the water exits the syringe. When the powder has become saturated with water after a short period of time, the plunger and associated piston 14 of the first syringe 10, which was initially in a locked condition, is now unlocked and immediately the plunger in the powder containing syringe is caused to advance by the remaining vacuum. This movement causes compaction of the now wet paste towards the distal end of the first syringe 10. Thumb pressure is now applied to the piston of the first syringe 10 which causes the hardenable bone cement paste to flow through the three-way tap connector and into the second syringe 20. Thumb pressure on the plunger of the second syringe 20 causes the hardenable bone cement paste to flow back into the first syringe 10. This movement of bone cement paste between the two syringes, backwards and forwards several times, mixes the cement to a homogeneous, de-aired condition.

With the hardenable bone cement, now in a homogeneously mixed and deaired condition in the first syringe 10, the second syringe 20 and three-way connector 30 can be unscrewed and discarded. An extension tube or cannula can now be screwed onto the male luer-lock 12 of the first syringe 10 and gentle thumb pressure applied to the plunger to extrude the de-aired cement from the syringe to the surgical site where it is allowed to set in-situ.

Calcium sulphate hemi-hydrate hardens and sets through hydration according to the following equation:

CaSO₄½H₂O+1½H₂O=CaSO₄2H₂O

### Example 2

In a second embodiment of the apparatus and method of the invention, which will now be described in relation to FIG. 4, a variation to the method and apparatus described in the first example is made by pre-combining the second syringe 20 and the three-way connector 30.

The powder component is supplied in a first syringe 10 in the same way as in the first embodiment. However, by supplying the second syringe 20 pre-connected to the three-way connector 30 with the water component contained both within the syringe 20 and the limb of the three-way connector 30 up-to the three-way valve the quantity of air trapped within the system can be minimised before de-airing.

In use, the end cap is first removed from the luer-lock end 12 of the powder containing syringe 10 which is then screwed onto the corresponding end of the three way tap in an air-tight manner. The evacuation and mixing procedures are then performed as described in Example 1 with the advantage that there is less air initially within the system.

### Example 3

A calcium phosphate powder mixture consisting of equimolar amounts of ground Ca4(PO4)2O (tetracalcium phosphate, TTCP) and CaHPO4 (dicalcium phosphate anhydrous, DCPA) was contained within a lockable piston syringe. The corresponding water component was contained within a simple piston syringe. The powder syringe was initially in the locked position. In use, the two syringes were connected together through a ‘T-piece’ connector and evacuation and mixing of the cement components was undertaken as previously described in Example 1.

### Example 4

An equal weight mixture of calcium sulphate alpha hemi-hydrate powder and beta tricalcium phosphate powder, having a maximum particle size of 150 microns, was prepared. To this mix was added 0.20% by weight of a high molecular weight hydroxypropyl methylcellulose powder (viscosity modifier). Five gram aliquots of the resulting mix were added to 10 ml capacity, lockable, piston syringes having a male luer-lock connector. A solution of tri-sodium orthophosphate decahydrate (a setting retarder) at a concentration of 0.50% was prepared and 1.80 ml aliquots were added to 5 ml capacity piston syringes having a female luer-lock connector. The components were deaired and mixed together as described in Example 1 above. The de-aired hardenable bone cement mixture was injectable through an 11's gauge needle of length 15 cm for a period of 9 minutes and it subsequently hardened within 40 minutes.

The de-aired hardenable bone cement, prepared as described in the preceding paragraph, was cast into cylindrical cavities in a silicone rubber mould to prepare cylindrical pieces suitable for compression testing. A comparable non de-aired hardenable bone cement material as described above was prepared in a similar manner except without the application of the vacuum/de-airing step. The comparable cement consequently contained entrapped air. The non de-aired cement was also cast into cylindrical cavities in a silicone rubber mould to prepare cylindrical pieces suitable for compression testing. Test pieces of each of the cements were allowed to set for 4 hours at room temperature prior to compression testing in a ‘wet’ condition. Further samples of each cement were dried at a temperature of 45 degrees centigrade overnight (16 hours) prior to compression testing in a ‘dry’ condition. Compression testing was undertaken, using a Zwick mechanical testing machine, along the cylindrical axis of the test pieces. The maximum force at failure was recorded. The results are shown in Table 1 and indicate a significantly higher compressive strength for both ‘wet’ and ‘dry’ material, with the de-aired bone cement produced by the method of the present invention.

TABLE 1

Compressive strength MPa

| | Non de-aired cement | De-aired cement |
|---|---|---|
| Wet strength | 6.3 +/− 1.0 | 10.8 +/− 0.6 |
| Dry strength | 9.4 +/− 1.1 | 24.4 +/− 0.8 |

Various modifications may be made without departing from the scope of the invention.

The syringes may be different to those indicated.

The three-way ‘T-piece’ connector or 3-way tap could be replaced by a 4-way tap with the fourth arm selectively connecting with the atmosphere. This would enable release of the vacuum without disconnecting the vacuum line.

The bone cement formulations may be different to those indicated.

The bone cement may comprise cations other than or in addition to calcium, such as sodium, potassium, magnesium, strontium or zinc.

The calcium sulphate may be in the form of beta hemi hydrate or soluble anhydrite.

In order to improve the ‘injectability’ of the de-aired hydraulic setting hardenable bone cement, a viscosity modifier or thickener may be included in the mixtures described above. This may be in the form of a powder added to the powder component of the mix or as a polymer powder pre-dissolved in the aqueous liquid component. The viscosity modifier or thickener may comprise, alone or in combination any of polyvinyl alcohol, polyethylene glycol, glycerol, carboxymethylcellulose, hydroxypropyl methyl cellulose, gelatine, hyaluronic acid, polyvinyl pyrrolidone, or other biocompatible polymer or viscous liquid.

The particle size of the component powder(s) may be different to that indicated.

Whilst endeavouring in the foregoing specification to draw attention to those features of the invention believed to be of particular importance, it should be understood that the Applicant claims protection in respect of any patentable feature or combination of features hereinbefore referred to and/or shown in the drawings whether or not particular emphasis has been placed thereon.

1. A method for preparing a de-aired hydraulic setting hardenable bone cement for filling a boney void or cavity by combining a powder bone substitute material and an aqueous liquid component, the method comprising the steps of:
(a) supplying said powder bone substitute material in a first evacuable container;
(b) withdrawing air from the first container to form a de-aired powder bone substitute material; and, then
(c) mixing the de-aired powder bone substitute material and the aqueous liquid component together.

2. The method of claim 1, wherein the mixing step (c) is performed at least partially within the first container.

3. The method of claim 1, wherein the mixing step (c) is performed under no touch conditions whereby the powder bone substitute material and the aqueous liquid component, are at all times kept isolated from external contamination during steps (b) and (c).

4. The method of claim 1, wherein the aqueous liquid component is provided in a second container and the mixing step (c) is performed by connecting the first and second containers together in fluid communication.

5. The method of claim 1, wherein air is withdrawn from the first container via a multi-port connector having a first port which is connected to an outlet of the first container and a second port which is connected to a vacuum source.

6. The method of claim 5, wherein said aqueous liquid component is supplied in a second container and wherein said multi-port connector has a third port which is connected to receive selectively the liquid component from an outlet of the second container.

7. The method as claimed in claim 5, wherein the multi-port connector is configured to permit selective fluid communication between the first container and the vacuum source and selective fluid communication between the first and second containers.

8. The method as claimed in claim 5, wherein during step (b) the outlet of said second container is blocked, whilst the outlet of the first container is connected to the vacuum source via the multi-port connector.

9. The method as claimed in claim 5, wherein once the first container has been evacuated to a desired low pressure state the connection between the vacuum source and the first container is closed to maintain the first container in said low pressure state.

10. The method as claimed in claim 9, wherein step (c) comprises connecting the first container in the desired low pressure state in fluid communication with the second container which is at a relatively higher pressure state such that the relative pressure difference causes liquid from the second container to be drawn into the first container.

11. The method as claimed in claim 10, wherein the first container is connected to the second container via said multi-port connector.

12. The method as claimed in claim 1, wherein said first container comprises a first syringe having a lockable piston and barrel construction.

13. The method of claim 12, wherein during said step (b) the piston of the first syringe is prevented from moving within said barrel and the pressure within the barrel of said first syringe is progressively reduced.

14. The method as claimed in claim 12, wherein during said step (b) said first syringe is positioned such that its' central axis is substantially horizontally aligned.

15. The method as claimed in claim 12, wherein the second container comprises a second syringe having a piston and barrel construction and the piston of said second syringe is free to move within its associated barrel, and the mixing step (c) is performed by connecting the first and second syringes together in fluid communication which causes the piston of the second syringe to advance as the liquid exits therefrom.

16. The method of claim 15, wherein following ingress of the liquid from the second syringe into the first syringe, the method further comprises switching the piston of said first syringe to an unlocked condition.

17. The method of claim 16, wherein the method further comprises reciprocating the de-aired hardenable bone cement between the first and second syringes by alternately depressing the plungers of the first and second syringes to cause the hardenable bone cement to pass between both syringes and progressively form a paste.

18. The method as claimed in claim 1, further including the step of dispensing the de-aired hardenable bone cement from said first and/or said second containers, preferably from said first container.

19. The method of claim 4, wherein the first and second containers, the multi-port connector and, optionally an air bleed tube for connection to a vacuum source, are supplied as a sterile kit of parts to an end user for configuration to connect the first and second containers and, optionally the air line, to the ports of the connector.

20. The method of claim 19, wherein the second container is supplied pre-connected to a port of the multi-port connector with the passage between the second container and the multi-port connector being pre-filled with liquid.

21. The method of claim 4, wherein the multi-port connector further includes a further port which is connectable to atmospheric pressure to allow easy removal of a connected vacuum source.

22. The method of claim 1, wherein the powder bone substitute material comprises a calcium salt based bone substitute material.

23. A method of filling a boney void or cavity in the skeletal system of a human or animal body, the method comprising forming a de-aired hydraulic setting hardenable bone cement by a method as claimed in claim 1, dispensing the de-aired hydraulic setting hardenable bone cement from said first and/or second container into a boney void or cavity of a human or animal body and allowing the de-aired hydraulic setting hardenable bone cement to set fully in-situ to form a bone cement.

24. Apparatus for the preparation of a de-aired hydraulic setting hardenable bone substitute material, in accordance with the method of claim 1, wherein the apparatus comprises a first evacuable container containing a powder bone substitute material, a second container containing a liquid component, and a multi-port connector having at least three ports and being connectable to an outlet of said first and second containers.

25. The apparatus of claim 24, wherein said first and second containers comprise first and second syringes of piston and barrel construction.

26. The apparatus of claim 25, wherein said first syringe includes means for locking said piston within said barrel.

27. The apparatus of claim 24, further comprising a source of vacuum.

28. The apparatus of claim 24, wherein said multi-port connector is configurable for one or more of the following states:
(a) to connect an outlet of the first container to a source of vacuum to permit reduction of the pressure within the first container to a given level below atmospheric pressure;
(b) to isolate the outlet of the first container from the source of vacuum thereby maintaining the first container in a reduced pressure state prior to connecting the outlet of the second container to the first container;
(c) to connect an outlet of the first container to an outlet of the second container, thereby permitting the aqueous liquid component to flow from the second container into the first container;
(d) to permit mixing of the de-aired powder bone substitute material and the aqueous liquid component within it, particularly by selective manipulation of piston plungers; and,
(e) to permit disconnection of the first container from the second container when the de-aired hydraulic setting hardenable bone cement is present in the first container.

29. The apparatus of claim 24, wherein the apparatus is supplied as a sterile kit of parts to an end user.

30. The apparatus of claim 24, wherein the multi-port connector is provided already connected to the outlet of the second container and the passage between them pre-filled with at least a part of the aqueous liquid component.

31. A de-aired hydraulic setting hardenable bone cement obtainable by the method as claimed in claim 1.
